(19) United States
(12) Patent Application Publication
(10) Pub. No.: US 2023/0158445 A1
(43) Pub. Date: May 25, 2023

(54) Process for Conversion of Bis(hydroxyethylethoxy)-urea to DGA

(71) Applicant: Huntsman Petrochemical LLC, The Woodlands, TX (US)

(72) Inventors: Patrick E Holub, The Woodlands, TX (US); Mark Forman, The Woodlands, TX (US)

(21) Appl. No.: 17/915,627

(22) PCT Filed: Mar. 31, 2021
(86) PCT No.: PCT/US21/25032

§ 371 Date: Sep. 29, 2022

Related U.S. Application Data
(60) Provisional application No. 63/003,664, filed on Apr. 1, 2020.
(60) Provisional application No. 63/003,959, filed on Apr. 2, 2020.

(51) Int. Cl. B01D 53/14; B01D 53/62

(52) U.S. Cl. CPC B01D 53/1425; B01D 53/1468; B01D 53/62; B01D 53/1475; B01D 2257/502; B01D 2257/504; B01D 2257/304; B01D 2257/308; B01D 2252/20447; B01D 2252/20484; B01D 2252/20489

(57) ABSTRACT

A reclaimer system and methods for using said reclaimer system to reclaim one or more amine agents from a fluid containing one or more degradation products that have been formed from the reaction of one or more acid gas components with the one or more amine agents.

### CROSS-REFERENCE TO RELATED APPLICATION

This application claims priority to United States Provisional Application Ser. Nos. 63/003,664 filed Apr. 1, 2020 and 63/003,959 filed Apr. 2, 2020. The noted applications are incorporated herein by reference.

### STATEMENT REGARDING FEDERALLY SPONSORED RESEARCH OR DEVELOPMENT

Not applicable.

### FIELD

The present disclosure generally relates to reclaimer systems and methods of using the reclaimer systems for converting bis(hydroxyethylethoxy)-urea to 2-2-aminoethoxyethanol.

### BACKGROUND

Gas treating processes for the removal of acid gas components often use amine-based compositions as the solvent. The processing units that are used in such processes circulate and contact the amine-based composition with the acid gas components allowing the amine to chemically react with the acid gas components. The amine-based composition is then regenerated to release the acid gas components and return the amine for reuse. One such amine that is commonly used in such processes is 2-2-aminoethoxyethanol, commercially available as DIGLYCOLAMINE® agent (DGA®) (Huntsman Petrochemical LLC). The use of diglycolamine in gas treating applications has been shown over the years to be quite valuable especially when used in demanding services.

Diglycolamine, in some gas treating applications, reacts with acid gas components CO₂ and COS to form bis(hydroxyethylethoxy)-urea, commonly referred to as BHEEU as shown below:

2DGA®agent+CO₂←→BHEEU+H₂O Reaction (1)

2DGA®agent+COS→BHEEU+H₂S Reaction (2)

where DGA® agent has the formula HOCH₂CH₂OCH₂CH₂—NH₂ and BHEEU has the
O
//
   formula HOCH₂CH₂OCH₂CH₂—NH—C—NH—CH₂CH₂OCH₂CH₂OH. Early in the history of use of diglycolamine in gas treating processes, it was discovered that Reaction (1) was reversible, and that BHEEU could be converted back into diglycolamine through the application of heat, time, and water.

Thus, while it was found that the formation of BHEEU was reversible under certain conditions, the reaction was generally not considered reversible at temperatures that were typically found in a reboiler and as such, historical design of gas treating processes using diglycolamine included a kettle-type thermal reclaimer that could be operated at low pressure (LP) and at a temperature of approximately 360° F.

With reference to FIG. 1, a process flow diagram is depicted illustrating such a conventional LP thermal reclaimer. The LP thermal reclaimer system was operated by:

(1) setting a fixed heat input into the system with an operating pressure generally of about 1 atmosphere pressure gauge;

(2) maintaining the fluid level within the vessel by the addition of amine-based composition to the vessel;

(3) controlling temperature within the vessel by the addition of water;

(4) allowing components which vaporized within the vessel at the above temperature and pressure to exit the top of the vessel (i.e. amine, water, CO₂ vapor); and

(5) periodically removing the non-boiling components which were retained in the vessel.

Most grass-roots gas treating processes which utilized diglycolamine were designed with this type of LP thermal reclaimer. LP thermal reclaiming was found to provide several process benefits, including removal of solids, removal of heat stable salts, removal of heavy oils and polymers and recovery of diglycolamine from BHEEU.

More recently, reclaimer systems utilizing diglycolamine have begun to operate at higher pressures to minimize the amount of water and amine that is vaporized while still providing adequate temperatures for BHEEU formation reversal. This design was presented at the LRGCC in 2010 showing the results of the first few applications of the technology. Operating pressures were estimated based on an attempt to maintain some vapor flow exiting the kettle-type reclaimer vessel (i.e. the key was to operate at a pressure where a portion of the CO₂ would be released to help drive the conversion of BHEEU back to diglycolamine). However, trying to determine this pressure was an estimated guess at best, and operators were left with a difficult task of trying to maintain and efficiently operate the system. Compounding this problem was the fact that daily operations were not “totally constant” and the composition of the amine-based composition would change throughout the operation as water was removed or added. Thus, the energy required to maintain the estimated operating pressure also changed with these changing compositions. In some cases, operating pressure was set too high and CO₂ would not vaporize, thus hindering the BHEEU reversal reaction. FIGS. 2 and 2a depict the process flow for this type of reclaimer system design which utilized a simple pressure control system to maintain the temperature of the fluid in the reclaimer vessel during its operation. This system operated by:

(1) setting a fixed flow of fluid into the vessel;

(2) maintaining the temperature of the fluid within the vessel by controlling the heat input;

(3) controlling operating pressure at a desired set point (generally, for example, at about 130 psig) using a pressure control valve; and

(4) maintaining the level of fluid within the vessel by a level control valve. The amount of vapor exiting the vessel was variable and based on how the system reacted to the fluid flow, heat input and pressure within the system.

It has been recognized the temperature of the fluid will vary significantly for this design which affects the amount of energy that is used during operation causing erratic operation. This variation in temperature also reduces the amount of diglycolamine reclaimed (e.g. when the temperature varies below the desired operational temperature), increases diglycolamine loss due to irreversible thermal degradation (e.g. when the temperature varies above the desired operational temperature). Thus, there is a continued need for systems and methods to increase the amount of diglycolamine recovered while reducing diglycolamine loss during reclaiming and a need for systems and methods that improve the stability of the temperature and energy requirements during operation of such systems.

### SUMMARY

The present disclosure provides a method for reclaiming one or more amine agents, the method comprising: controlling temperature of a fluid in a reclaimer vessel by allowing a fixed amount (e.g. volume) of vapor output stream to exit the reclaimer vessel while allowing pressure within the reclaimer vessel to vary wherein the fluid comprises one or more degradation products that have been formed from reaction of the one or more amine agents with one or more acid gas components and the vapor comprises CO₂ and amine agent. In one particular embodiment, the amine agent comprises diglycolamine and the degradation product comprises BHEEU. In other embodiments, the temperature of the fluid within the reclaimer vessel is controlled such that it is maintained within about 5% (e.g., 3%, 2%, 1%, 0.75%, 0.5%, or 0.2%) of the desired temperature (e.g. about 360° F.) or desired temperature range (e.g. about 355°−385° F.).

The present disclosure also provides a system for reclaiming one or more amine agents, the system comprising: (i) a reclaimer vessel comprising a fluid, wherein the fluid comprises one or more degradation products that have been formed from reaction of the one or more amine agents with one or more acid gas components; (ii) a steam input subsystem configured to provide energy into the fluid in the reclaimer vessel; (iii) a fluid input subsystem configured to provide the fluid to the reclaimer vessel; (iv) a level control subsystem configured to control the amount of fluid within the reclaimer vessel and (v) a vapor outlet subsystem configured to control an amount (e.g. volume) of vapor exiting the vessel, wherein temperature of the fluid within the reclaimer vessel is maintained at a desired temperature or temperature range by fixing the amount (e.g. volume) of vapor exiting the vessel while allowing pressure within the reclaimer vessel to vary.

Accordingly, the systems and methods disclosed herein may be used to convert any degradation products reversibly formed from one or more amine agents into reclaimed, useable amine agent. Such amine agents, in addition to diglycolamine, may include, but are not limited to, monoethanolamine (MEA), diethanolamine (DEA), diisopropanolamine (DIPA), triethanolamine (TEA), methyldiethanolamine (MDEA), 2-amino-2-methyl-1-propanol (AMP), piperazine (PZ), and combinations thereof. The systems and methods disclosed herein may be used for post-combustion capture, gas sweetening, or any other process where degradation products are formed from reactions of amine agents and acid gas components and amine agent losses are undesirable.

### BRIEF DESCRIPTION OF THE DRAWINGS

FIG. 1 depicts a process flow diagram for a conventional low pressure (LP) thermal reclaimer system;

FIG. 2 depicts a process flow diagram for conventional reclaimer systems utilizing a pressure control system;

FIG. 2A depicts an enlarged view of the vapor outlet control system (Section I) of the conventional reclaimer system as illustrated in FIG. 2;

FIG. 3 depicts a process flow diagram for a reclaimer system according to embodiments of the present disclosure utilizing a vapor outlet control system;

FIG. 3A depicts an enlarged view of the vapor outlet control system (Section II) of the reclaimer system of FIG. 3; and

FIGS. 4 and 5 are plots illustrating the relative vapor off flash reclaimer v. relative operating pressure of the reclaimer system at various weight percentages.

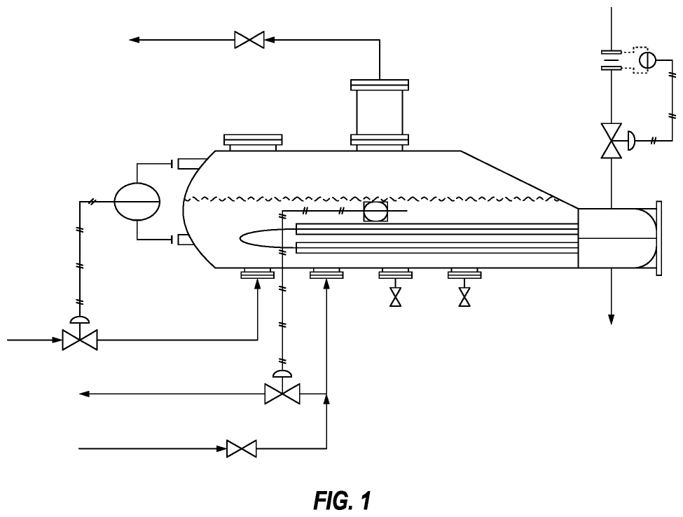

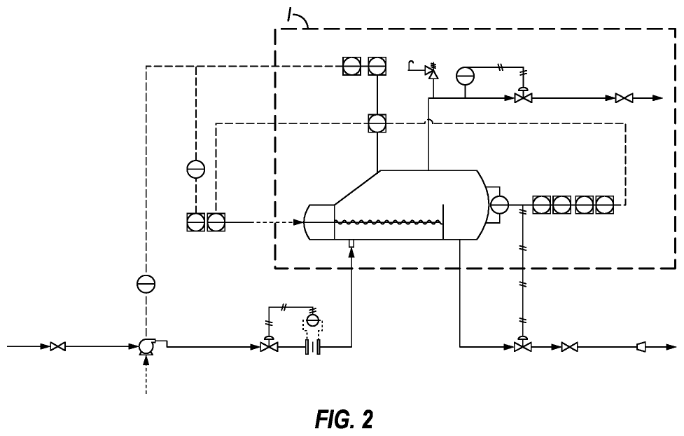

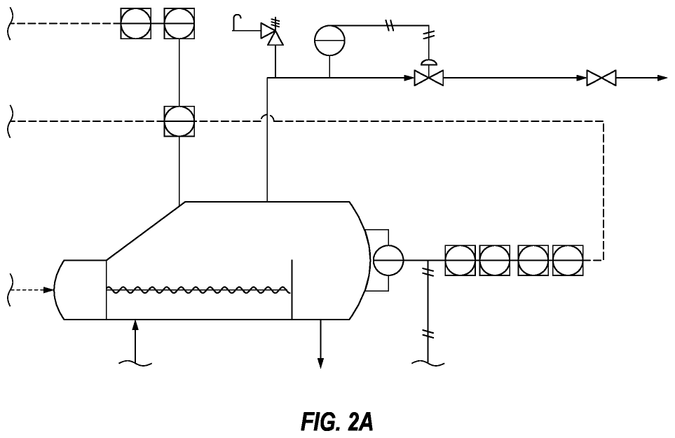

### DETAILED DESCRIPTION

The following terms shall have the following meanings:

The term “comprising” and derivatives thereof are not intended to exclude the presence of any additional component, step or procedure, whether or not the same is disclosed herein. In order to avoid any doubt, all compositions claimed herein through use of the term “comprising” may include any additional additive or compound, unless stated to the contrary. In contrast, the term, “consisting essentially of” if appearing herein, excludes from the scope of any succeeding recitation any other component, step or procedure, except those that are not essential to operability and the term “consisting of”, if used, excludes any component, step or procedure not specifically delineated or listed. The term “or”, unless stated otherwise, refers to the listed members individually as well as in any combination.

The articles “a” and “an” are used herein to refer to one or to more than one (i.e. to at least one) of the grammatical objects of the article. By way of example, “an amine agent” means one amine agent or more than one amine agent. The phrases “in one embodiment”, “according to one embodiment” and the like generally mean the particular feature, structure, or characteristic following the phrase is included in at least one embodiment of the present disclosure, and may be included in more than one embodiment of the present disclosure. Importantly, such phrases do not necessarily refer to the same aspect. If the specification states a component or feature “may”, “can”, “could”, or “might” be included or have a characteristic, that particular component or feature is not required to be included or have the characteristic.

The term “about” as used herein can allow for a degree of variability in a value or range, for example, it may be within 10%, within 5%, within 1%, within 0.75%, within 0.5%, within 0.25% or within 0.1% of a stated value or of a stated limit of a range.

The term “amine agent” refers to a chemical species comprising an amine functional group. An amine agent can be a primary amine, secondary amine, or tertiary amine, or a combination thereof. In at least one example, the amine agents can be a cyclic diamine. In an alternative example, the amine agents may be an alkanolamine. Amine agents suitable for use with the methods and systems disclosed herein include, but are not limited to, diglycolamine (DGA), monoethanolamine (MEA), diethanolamine (DEA), diisopropanolamine (DIPA), triethanolamine (TEA), methyldiethanolamine (MDEA), 2-amino-2-methyl-1-propanol (AMP), piperazine (PZ), and combinations thereof. For example, one or more amine agents may be used in a single process (e.g., gas sweetening process) such that at least some of each of the one or more amine agents is reclaimed with the reclaimer system.

The term “fluid” is used herein as is conventional in the art when referring to reclaimers. Namely, “fluid” comprises at least some amount of one or more degradation products and can further comprise another solvent or species including, but not limited to, water.

The term “degradation product” refers to chemical species that form an amine agent. It will be appreciated that degradation products as a term of art may include, for example, both products of a reaction of an amine agent and another species (an acid gas component including, without limitation, carbon dioxide (CO₂), carbonyl sulfide (COS), carbon monoxide (CO), hydrogen sulfide (H₂S), and carbon disulfide (CS₂)), as well as thermal degradant byproducts that may form during reclamation of the amine agent. Degradation products may reversibly or irreversibly be formed. As used herein, heat stable salts that comprise degraded amine agents may also be degradation products.

The term “operational cycle” is used to refer to the period of time a reclaimer system is actively being used to reclaim useable amine agent from degradation products. The operational cycle may start when a reclaimer vessel is initially filled with the fluid and ends when the reclaimer vessel is flushed to remove byproducts accumulated in the bottom of the reclaimer vessel.

The terms “desired temperature” and “desired temperature range” refer to a pre-selected temperature or temperature range at which a reclaimer system is configured to operate. Generally, the desired temperature or temperature range for a reclaimer system or method of its use depends on the amine agent being used. The desired temperature or temperature range for a reclaimer system or method may be selected in order to limit irreversible thermal degradation of an amine agent below a threshold rate of thermal degradation. For example, in certain embodiments, when a system or method uses diglycolamine as its sole amine agent, the desired temperature can be a range of from about 355°−385° F. In at least one example, the desired temperature range from about 355°−365° F. In an additional example, the desired temperature can be about 360° F. In certain embodiments, a desired temperature or temperature range is used as a temperature set point for a reclaimer system.

The present disclosure herein provides a system for reclaiming an amine agent from a fluid with a reclaimer system that improves the temperature stability of fluid and energy requirements for the system while reclaiming the amine agent. The present disclosure also provides methods of use for such systems. The system generally comprises a reclaimer vessel, a fluid input subsystem, a steam input subsystem, a level control subsystem and a vapor output subsystem that is configured to control the amount (e.g. volume) of vapor exiting the vessel in order to maintain the temperature of the fluid in the reclaimer vessel. As one skilled in the art is aware, the energy requirement to maintain the proper operating conditions in a reclaimer vessel in order to reclaim the amine agent, such as diglycolamine, is determined by several factors including, without limitation: (i) the composition of the fluid entering the reclaimer vessel; (ii) the sensible heat required to raise the temperature of the fluid to the operating conditions, (iii) the heat of reaction required to reverse the reaction of BHEEU; (iv) the heat of reaction required to reverse the CO₂/DGA reaction; and (v) the heat of vaporization for any vapors exiting the reclaimer vessel. For a given fluid rate into the vessel, the largest variable that contributes to changes in the energy requirement is the heat of vaporization for vapors exiting the reclaimer vessel. Applicant has surprisingly found that by controlling the amount (e.g. volume) of vapors exiting the reclaimer vessel while allowing the pressure to change, a desired operational temperature (and therefore a more consistent heat input) can be maintained during the operational cycle. Since the energy requirement is mainly determined by the temperature rise (sensible heat) and vaporization energy (a fixed value), the total energy required for this system is not severely impacted by fluctuations of the pressure and minor changes in the composition of the feed as for conventional systems. Moreover, controlling the amount of vapor exiting the reclaimer vessel improves residence time of the fluid within the reclaimer vessel thereby improving the amount of amine agent that is recovered.

Thus, in certain embodiments, the present disclosure includes a method for reclaiming one or more amine agents (e.g. DGA), the method comprising: controlling temperature of a fluid in a reclaimer vessel (e.g., a horizontal or vertical reclaimer vessel), at least in part, by allowing a fixed amount (e.g. volume) of vapor output stream to exit the reclaimer vessel while allowing pressure within the reclaimer vessel to vary, where the fluid comprises one or more degradation products (e.g. BHEEU) that have been formed from reaction of the one or more amine agents (e.g. diglycolamine) with one or more acid gas components (e.g. CO₂) and where the vapor comprises CO₂ and amine agent.

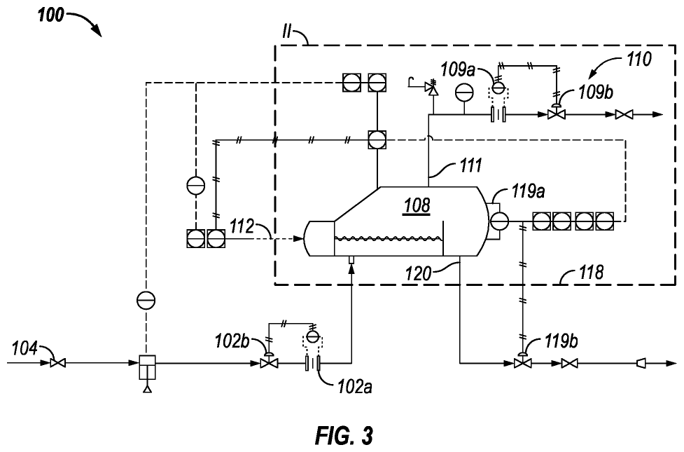

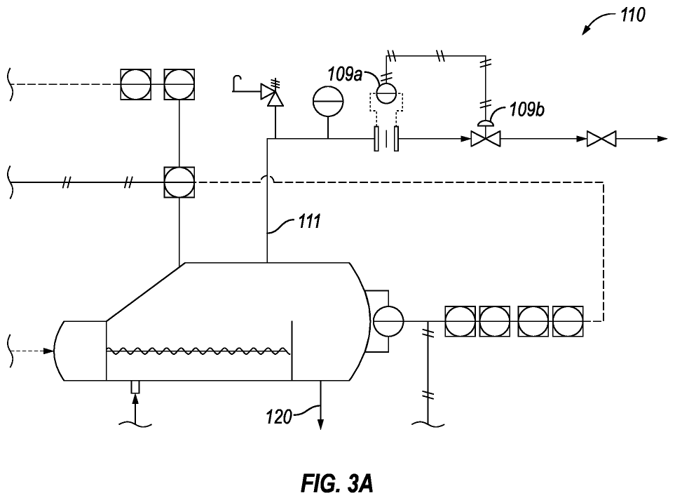

With reference now to FIGS. 3 and 3a, a process flow diagram for an exemplary reclaimer system 100 according to the present disclosure is shown. Such a reclaimer system may be used as part of many gas sweetening systems currently in operation throughout the world. The fluid from which the amine agent is reclaimed in system 100 is contained in vessel 108. During operation, in order to heat the fluid to the desired operational temperature, steam is input into vessel 108 using steam input subsystem 112. Steam flows through subsystem 112 into vessel 108 where it is in indirect contact with fluid in the vessel 108. Steam input subsystem 112 may include one or more tube bundles through which the steam flows that are in contact with the fluid in the vessel 108. Alternatively, steam may be input directly into a vessel. Steam input subsystem 112 is configured to provide a constant input of steam during the operational cycle. Steam flow rate and steam temperature are predetermined based on the desired temperature or temperature range of the reclaimer system. In some embodiments, the fluid may heated by other means known to those skilled in the art, such as, but not limited to, through the use of hot oil or direct fired heaters.

Fluid is provided to the vessel 108 by the fluid input subsystem 104. Fluid input subsystem 104 comprises flow indicator and controller 102a and control valve 102b that collectively control the amount of fluid that flows through the subsystem into vessel 108.

The level of fluid within the vessel 108 is controlled by level control subsystem 118. Level control subsystem 118 includes control valve 119a and level controller 119b which collectively maintains the level of fluid within the vessel 108.

The temperature of the fluid within vessel 108 is controlled (i.e. maintained stably at the desired temperature or temperature range) by the vapor output subsystem 110. Vapor output subsystem 110 includes outlet 111, flow indicator and controller 109a and control valve 109b which collectively controls the amount (i.e. volume) of vapor including CO₂ and amine agent that exits outlet 111 from vessel 108 and flows through variable vapor output system 110. As can be seen in FIG. 3, the steam flow through steam input subsystem 112, fluid flow through fluid input subsystem 104 and operating pressure are independent of the vapor output subsystem 110. Controlling the amount (e.g. volume) of vapor exiting the vessel 108 aids in stabilizing the reclaimer system 100 by making the main heat input requirements (sensible heat and heat of vaporization) more constant variables which therefore allows for more steady operations and fewer fluctuations in the operational cycle.

Throughout operation of the reclaimer system 100 during an operational cycle, steam input constantly heats fluid in vessel 108, operating pressure varies, vapor output subsystem 110 controls the temperature of the fluid by fixing the amount (e.g. volume) of vapor exiting vessel 108, and fluid input subsystem 104 and level control subsystem 118 maintains the level of fluid within vessel 108. In at least one example, water, CO₂ and amine agent vapors form due to the elevated temperature within vessel 108 and exit vessel 108 through outlet 111 of the vapor output system 110. Outlet 111 is located on the top of vessel 108 in order to allow the vapors to naturally escape. Outlet 120 is disposed on the bottom of vessel 108 in order to allow remaining fluid back to the reclaimer system 100. Unlike conventional LP reclaimer systems, reclaimer system 100 operates continuously so that vessel 108 does not need to be dumped/flushed therefore eliminating waste and the need for operator intervention.

Vessel 108 is depicted as a horizontal vessel. Thus, in certain embodiments, a horizontal vessel is used. Alternatively, in certain embodiments, a vertical vessel may be used. One or more auxiliary vessels may be included in reclaimer system 100 in addition to a main reclaiming vessel 108. In certain embodiments, vessel 108 is a conventional horizontal kettle reclaimer. Vessels with a range of length to diameter ratios (L/D ratios) can be used. For example, vessel 108 can have an L/D ratio of between about 2 and about 5, between about 1 and about 4, between about 2 and about 6, less than about 5, less than about 4, less than about 3, or less than about 2. Reclaimer vessel 108 may be operated at a positive pressure which, as discussed above, is allowed to vary during the operational cycle.

According to another embodiment, a method comprises controlling the volume of vapor exiting a reclaimer vessel during an operational cycle at a fixed amount while allowing pressure within the reclaimer vessel to vary to thereby control temperature of a fluid comprising one or more amine agents within the reclaimer vessel at a desired temperature or within a desired temperature range whereby thermal degradation of the one or more amine agents is inhibited during the operational cycle (e.g., wherein the desired temperature or desired temperature range has been determined based on thermal degradation temperature(s) of at least one of the one or more amine agents). In certain embodiments, temperature is controlled such that it is maintained within about 5% (e.g., about 3%, 2%, 1%, 0.75%, 0.5%, or 0.2%) of the desired temperature or the desired temperature range. In certain embodiments of systems and methods disclosed herein, the one or more amine agents comprises diglycolamine and the amount of vapor exiting the vessel is fixed during an operational cycle in order to maintain the temperature within between about 355° F. and about 385° F. (e.g., between about 355° and about 365° F., between about 358° F. and about 362° F.). In certain embodiments of systems and methods disclosed herein, the temperature is maintained at about 360° F. (e.g., within about 0.75%, 0.5%, or 0.2% of 360° F.).

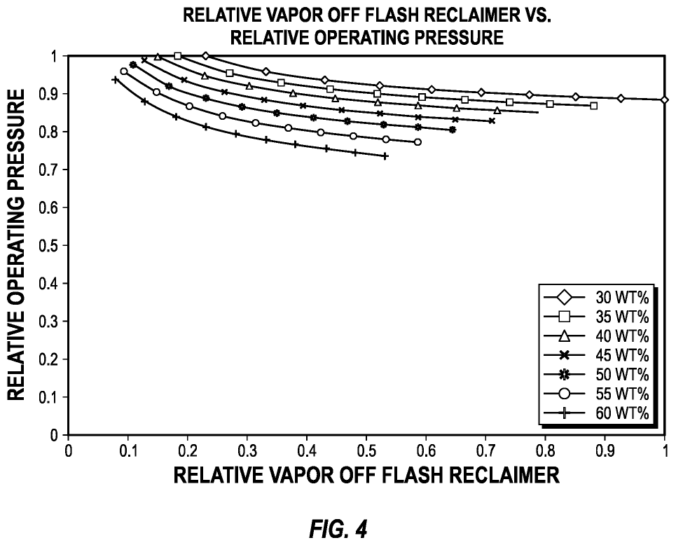

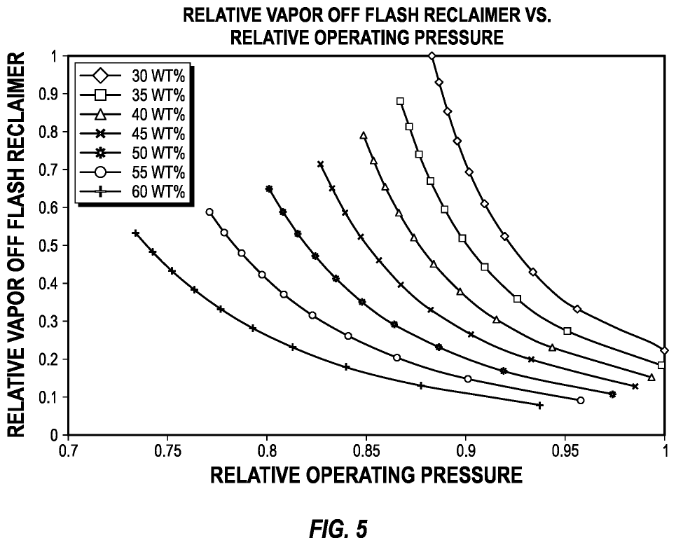

FIGS. 4 and 5 illustrate relative vapor off flash reclaimer vs. relative operating pressure plots at various weight percentages. Specifically, the relative vapor off flash reclaim is monitored with respect to the relative operating pressure within the reclaimer vessel.

While the foregoing is directed to various embodiments of the present disclosure, other and further embodiments of the disclosure may be devised without departing from the basic scope thereof, and the scope thereof is determined by the claims that follow.

What is claimed is:

1. A method for reclaiming one or more amine agents, the method comprising:
controlling a temperature of a fluid in a reclaimer vessel by allowing a fixed amount of a vapor output stream to exit the reclaimer vessel while allowing pressure within the reclaimer vessel to vary,
wherein the fluid comprises one or more degradation products that have been formed from reaction of one or more amine agents with one or more acid gas components and the vapor output stream comprises one or more amine agents.

2. The method of claim 1, wherein at least one of the one or more degradation products of the fluid is bis(hydroxyethylethoxy)-urea (BHEEU).

3. The method of claim 1, wherein the one or more amine agents are selected from the group consisting of: 2-2-aminoethoxyethanol, monoethanolamine (MEA), diethanolamine (DEA), diisopropanolamine (DIPA), triethanolamine (TEA), methyldiethanolamine (MDEA), 2-amino-2-methyl-1-propanol (AMP), piperazine (PZ), and combinations thereof.

4. The method of claim 1, wherein the one or more acid gas components are selected from the group consisting of: carbon dioxide (CO₂), carbonyl sulfide (COS), carbon monoxide (CO), hydrogen sulfide (H₂S), carbon disulfide (CS₂), and combinations thereof.

5. The method of claim 4, wherein the vapor output stream further comprises carbon dioxide (CO₂).

6. The method of claim 1, wherein the temperature of the fluid is maintained between about 355° F. to about 385° F.

7. A system for reclaiming one or more amine agents, the system comprising:
a reclaimer vessel comprising a fluid, wherein the fluid comprises one or more degradation products formed by a reaction of one or more amine agents with one or more acid gas components;
a steam input subsystem for providing energy into the fluid in the reclaimer vessel;
a fluid input subsystem for controlling a rate at which the fluid enters the reclaimer vessel;
a level control subsystem for controlling a level of fluid within the reclaimer vessel; and
a vapor outlet subsystem for controlling an amount of a vapor exiting the reclaimer vessel,
wherein a temperature of the fluid within the reclaimer vessel is maintained by fixing the amount of the vapor exiting the reclaimer vessel.

8. The system of claim 7, wherein the amount of the vapor exiting the reclaimer vessel is fixed during an operational cycle.

9. The system of claim 7, wherein the temperature of the fluid in the reclaimer vessel is maintained between about 355° F. to about 385° F.

10. The system of claim 7, wherein a pressure within the reclaimer vessel varies.

11. The system of claim 7, wherein the one or more degradation products are generated by the reaction of the one or amine agents and the one or more acid gas components.

12. The system of claim 7, wherein at least one of the one or more degradation products of the fluid is bis(hydroxyethylethoxy)-urea (BHEEU).

13. The system of claim 7, wherein the one or more amine agents are selected from the group consisting of: 2-2-aminoethoxyethanol, monoethanolamine (MEA), diethanolamine (DEA), diisopropanolamine (DIPA), triethanolamine (TEA), methyldiethanolamine (MDEA), 2-amino-2-methyl-1-propanol (AMP), piperazine (PZ), and combinations thereof.

14. The system of claim 7, wherein the one or more acid gas components are selected from the group consisting of: carbon dioxide (CO₂), carbonyl sulfide (COS), carbon monoxide (CO), hydrogen sulfide (H₂S), carbon disulfide (CS₂), and combinations thereof.

15. The system of claim 14, wherein the vapor output stream further comprises carbon dioxide (CO₂).
